(19) United States
(12) Patent Application Publication
(10) Pub. No.: US 2021/0085703 A1
(43) Pub. Date: Mar. 25, 2021

(54) Compositions and Methods for Preventing or Treating Nephrolithiasis

(71) Applicant: SP Nutraceuticals, Inc., Oakville (CA)

(72) Inventors: Paul Spagnuolo, Oakville (CA); Thamara DAYARATHNA, London (CA); Hon Sing LEONG, Rochester, MN (US)

(21) Appl. No.: 16/630,617

(22) PCT Filed: Jul. 10, 2018
(86) PCT No.: PCT/CA2018/000139

§ 371 Date: Jan. 13, 2020

Related U.S. Application Data
(60) Provisional application No. 62/532,679, filed on Jul. 14, 2017.

(51) Int. Cl. A61K 31/7034; A61K 49/00; A61K 31/407; A61K 31/167; A61K 31/192; A61K 31/616; A61K 45/06; A61K 33/06; A61K 33/00; A61K 31/717; A61K 31/18; A61K 31/197; A61P 13/04; G01N 33/58; G01N 33/50

(52) U.S. Cl. CPC A61K 31/7034; G01N 2800/347; A61K 31/407; A61K 31/167; A61K 31/192; A61K 31/616; A61K 45/06; A61K 33/06; A61K 33/00; A61K 31/717; A61K 31/18; A61K 31/197; A61P 13/04; G01N 33/582; G01N 33/5085; G01N 2800/52; A61K 49/0008

(57) ABSTRACT

The invention relates to the use of a compound having the structure of Formula (I) or a pharmaceutically acceptable salt or solvate thereof, for treating or preventing nephrolithiasis in patients. The invention further relates to an assay to identify patients likely to benefit from administration of a compound of Formula (I). The invention also relates to an assay to identify putative anti-lithogenic agents.

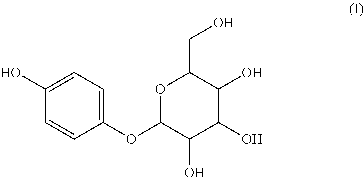 [SMILES: OCC1OC(Oc2ccc(O)cc2)C(O)C(O)C1O]

### FIELD

This invention relates generally to methods and compositions for preventing or treating formation or presence of nephroliths in mammals. Particularly, the disclosure relates to the methods and compositions comprising hydroquinone β-D-glucopyranoside of Formula (I) alone or optionally in combination with another agent for treating or preventing nephroliths in a subject.

### BACKGROUND

Nephrolithiasis, generally known as formation of urinary stones, is a common urological disorder affecting more than 10% of the population in industrialized nations. In the United States, the prevalence of stone disease increased from 5.2% (1994) to 8.4% (2012). Globally, the incidence and prevalence of nephrolithiasis demonstrates a similar trend and contributes significantly to the development of chronic kidney disease (CKD).

Hydroquinone β-D-glucopyranoside of Formula (I), also known as arbutin, is found in the dried leaves of a number of different plant species including bearberry (Arctostaphylos uvaursi):

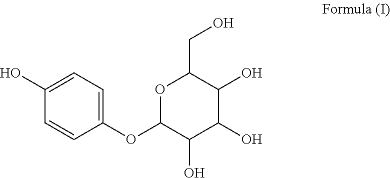 [SMILES: OCC1OC(Oc2ccc(O)cc2)C(O)C(O)C1O]

Arbutin has been reported to have antimicrobial and disinfectant properties, as an inhibitor of melanin formation and it has been used in some skin-lightening products. For example, U.S. Pat. No. 9,168,398, describes arbutin-containing compositions for topical application to provide enhanced luminosity, brightening or lightening to the skin of a user. WO2014040632 describes a composition or dietary supplement comprising a synergistic association of arbutin and another plant extract, berberine, for improving the health of the urinary tract and preventing or treating acute and recurrent urinary tract infections (UTI).

The direct costs of treatment and indirect costs of productivity time lost to nephrolithiasis exceeded $5.3 billion in 2000 in the United States alone. The complex process of nephrolith formation can be the result of a cascade of events, including crystal nucleation, growth and aggregation, and crystal retention within the renal tubules. Several urinary proteins play important roles in nephrolithiasis. Despite sizeable nephrolithiasis related costs and morbidity, with 5-year recurrence rates approaching 50% in affected individuals, a complete understanding of nephrolithiasis on the molecular level remains unclear.

The pathogenesis of calcium oxalate nephrolithiasis, the most common stone subtype (80%), is multifactorial. Predisposing factors include metabolic disorders such as hypercalcuria, hyperoxaluria or hypocitraturia and environmental factors, for example, diet. These etiological elements disturb the metastable biochemical homeostasis of urine which ultimately culminates in crystal deposition and stone formation.

Methods for treatment of nephrolithiasis have focused on surgical removal of stones or dietary adjustments in some cases. These treatment options have inherent disadvantages. For example, a patient must be limited more or less completely to a specific food or reduce intake of essential minerals, which may have deleterious effects on the patient's long-term health. The medical option of extracorporeal lithotripsy uses high-energy shock waves to fragment and disintegrate kidney stones, which is an expensive procedure. In addition, lithotripsy can cause blood in the urine and is only effective for the treatment of large kidney stones.

Medical treatment and prevention strategies for calcium oxalate based nephroliths, which have remained relatively stagnant since the mid-1980's, vary based on underlying etiology. In primary and idiopathic hyperoxaluria, which can also lead to calcium oxalate nephrolith formation, evidence is sparse for a consistently effective medical treatment. Pyridoxine has been suggested in the past based on several small non-randomized clinical trials. Newer approaches such as probiotic and oxalate decarboxylase treatment have generated mixed results.

Accordingly, there is a need for an efficacious, cost effective, and non-surgical therapeutic agent and/or treatment option for treating and preventing kidney stones and/or gallstones and symptoms associated with kidney stones and/or gallstones. There is an additional need for therapeutic agent and/or treatment protocol which can reduce the rate of or prevent the formation of calculi in the kidneys and/or gallbladder.

### SUMMARY

Described herein are methods and compositions for preventing or treating formation or presence of nephroliths in mammals.

Accordingly, an aspect of the disclosure is use of a compound having the structure of Formula (I):

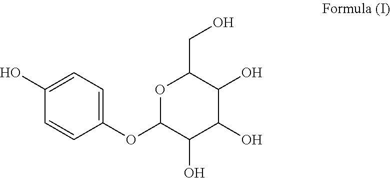 [SMILES: OCC1OC(Oc2ccc(O)cc2)C(O)C(O)C1O]

or a pharmaceutically acceptable salt or solvate thereof, for reducing the size and/or number of calcium oxalate based nephroliths in a subject in need thereof.

In an embodiment, the compound of Formula (I) or a pharmaceutically acceptable salt or solvate thereof is a compound that is used for treating or preventing nephrolithiasis in the subject.

Another aspect of the disclosure is the use wherein hydroquinone β-D-glucopyranoside or pharmaceutically acceptable salt or solvate thereof is comprised in a pharmaceutical composition.

In an embodiment, the pharmaceutical composition comprises one or more suitable excipients, diluents, buffers, carriers or vehicles.

In yet another embodiment, the pharmaceutical composition is in a solid, liquid, oral or injectable dosage form.

In yet another embodiment, the pharmaceutical composition is administered by parenteral, subcutaneous, intravenous, intraperitoneal, transdermal, oral, buccal, intravaginal, intravesicular, depot injection or implants.

Another aspect of the disclosure is a combination comprising a pharmaceutical composition comprising a compound of Formula (I) and/or a pharmaceutically acceptable salts or solvates thereof and an additional agent.

In one embodiment, hydroquinone β-D-glucopyranoside is administered with an additional agent selected from the group consisting of ketorolac, acetaminophen, ibuprofen, aspirin, xanthine oxidase inhibitor, potassium citrate, potassium magnesium, magnesium citrate, neutral (nonacidic) sodium, potassium phosphate, cellulose phosphate, cholestyramine, tamsulosin, tiopronin, diuretics, hydrochlorothiazide, chlorothiazide, trichlormethiazide, chlorthalidone, amiloride, citrate salts, phosphates, cholestyramine, sodium bicarbonate, aluminum hydroxide anti-acid gel, acetohydroxamic acid, allopurinol, penicillamine, captopril, nonsteroidal anti-inflammatory drugs (NSAIDs) and any combination thereof.

Another aspect of the disclosure is the use of a compound of Formula (I) and/or a pharmaceutically acceptable salt or solvate thereof for treating or preventing symptoms associated with nephrolithiasis in a subject.

In one embodiment, the symptom treated or prevented by the use of hydroquinone β-D-glucopyranoside or a pharmaceutically acceptable salt or solvate thereof is selected from any of the following: pain, fever, chills, blood in urine, hypercalcemia, hyperthyroidism, hyperparathyroidism, sarcoidosis, sjogrens syndrome, crohns disease, insulin resistance, acquired renal tubular acidosis, gout, osteoporosis, osteopenia, obesity, overweight, hypertension, anorexia/bulimia, malignancy, urinary dysfunction, abnormal urine odor, urinary leakage; urinary incontinence, urinary leakage, urinary hesitancy, weak urination, urinary blockage, urinary dribbling, nocturnal enuresis, urinary urgency, increased urinary frequency and any combination thereof.

In one embodiment, the amount of hydroquinone β-D-glucopyranoside or a pharmaceutically acceptable salt or solvate thereof ranges from about 50 mg to 850 mg/day.

Another aspect of the disclosure is a test assay for identifying putative anti-lithogenic agent comprising:
a. inducing a screenably distinct characteristic in wild-type Drosophila by feeding a modified diet;
b. feeding to the Drosophila a compound that putatively modifies the screenably distinct characteristic; and
c. screening and imaging the Drosophila to determine whether the compound modifies the screenably distinct characteristic.

In one embodiment, the screenably distinct characteristic in the test assay for identifying putative anti-lithogenic agent comprises formation of calcium oxalate based nephroliths.

In another embodiment, the modified diet in the test assay for identifying putative anti-lithogenic agent comprises stone forming media with sodium oxalate.

In yet another embodiment, the test assay for identifying putative anti-lithogenic agent further comprises screening the Drosophila to determine whether the compound has a toxic effect on the Drosophila.

Another aspect of the disclosure is a method of identifying a subject with nephrolithiasis likely to benefit from administration of a comnound of Formula (I) having the structure

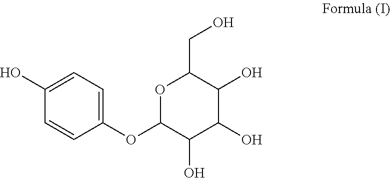 [SMILES: OCC1OC(Oc2ccc(O)cc2)C(O)C(O)C1O]

or a pharmaceutically acceptable salt or solvate thereof, comprising:
a. Obtaining a test sample comprising urine sample from the subject;
b. Determining the calcium oxalate stone burden of the test sample; and
c. Comparing the stone burden of the test sample to urine sample of a control;
   wherein the subject is identified as likely to benefit from the administration of the compound of Formula (I) or pharmaceutically acceptable salt or solvate thereof when the urine sample has an at least 2 fold increased calcium oxalate stone burden compared to the control.

Another aspect of the disclosure is a kit comprising a compound of Formula (I) having the structure

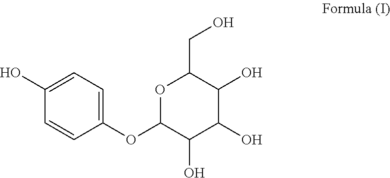 [SMILES: OCC1OC(Oc2ccc(O)cc2)C(O)C(O)C1O]

or a pharmaceutically acceptable salt or solvate thereof, optionally another agent, and/or packaging instructions for use thereof.

Other features and advantages of the present disclosure will become apparent from the following detailed description. It should be understood, however, that the detailed description and the specific examples while indicating preferred embodiments of the disclosure are given by way of illustration only, since various changes and modifications within the spirt and scope of the disclosure will become apparent to those skilled in the art from this detailed description.

### BRIEF DESCRIPTION OF THE DRAWINGS

An embodiment of the present disclosure will now be described in relation to the drawings in which:

FIG. 1 shows calcium oxalate calculi formation in D. melanogaster according to an embodiment of the present invention. FIG. 1a) Confocal and birefringence images of pulverized human calcium oxalate crystals (upper row) and synthetic hydroxyapatite particles (lower row) stained with Alendronate-FITC probe. FIG. 1b) Schematic of D. melanogaster model of calcium oxalate calculi formation within Malpighian Tubules (MTs). FIG. 1c) Intravital imaging of RFP expressing diet induced calcium oxalate stone within MTs of D. melanogaster larvae. Arrow indicates an RFP+ve MT with birefringent signal (c, bottom panel). FIG. 1d) Dissected MTs reveal the presence of Alendronate-FITC positive deposits (white arrows) within RFP MTs, confirming the presence of oxalate-based calculi. FIG. 1e) Dissected MTs as imaged by brightfield or scanning electron microscopy (SEM). FIG. 1f) SEM/EDX analysis of calcium oxalate monohydrate calculi extracted from MTs. FIG. 1g) SEM/EDX analysis of calcium oxalate dehydrate calculi extracted from MTs.

FIG. 2 shows the presence of calculi in fecal excreta of D. melanogaster according to another embodiment of the present invention. FIG. 2a) Increasing amounts of sodium oxalate in fly media induced kidney stone formation, present within MTs as evidenced by birefringence signal and Alendonrate-FITC signal. FIG. 2b) D. melanogaster survival curve on sodium oxalate treated fly media over a 60-day period. FIG. 2c) D. melanogaster deposit calculi-rich fecal excreta on the fly wall and coverslip attached to sponge lid. Flies grown in sodium oxalate rich fly media produce fecal excrement containing birefringent bodies representing calculi. FIG. 2d) Schematic of in vivo drug library screening for anti-lithogenic compounds based on the calculi-fecal excreta coverslip assay. FIG. 2e) Drug screening results from a chemical library representing 360 compounds and screened in duplicate. Hit was defined as coverslips that yielded a <20% reduction in calculi deposition in fecal excreta. Thresholds for inhibition were selected using in vitro analysis with synthetic calcium oxalate crystals. Confirmation using dose-dependent analysis in vivo led to arbutin.

FIG. 3 shows arbutin and its anti-lithogenic effects on oxalate-based calculi according to another embodiment of the present invention. FIG. 3a) Polarized microscopy of MTs treated with arbutin compared to standard media and oxalate supplemented fly media. FIG. 3b) Percentage of fecal excreta area that contains birefringence signal/calculi when flies are grown in various fly media treatments, including arbutin. * denotes p<0.01 with two-way ANOVA; Scheffe α correction. FIG. 3c) Dose dependent inhibition of oxalate-based fecal excreta deposited by D. melanogaster with various concentrations of arbutin. FIG. 3d) Microscopy of coverslips deposited with calculi-rich fecal excreta and dissected MTs when fly media is supplemented with 0, 32, and 512 μM arbutin. FIG. 3e) Schematic of patient urine based kidney stone in vivo formation assay. Patient urine samples are added to fly media and no other supplement. Coverslips containing calculi-rich fecal excreta are analyzed via microscopy. FIG. 3f) The patient urine based kidney stone in vivo formation assay was used to compare potassium citrate with arbutin at various pHs. Coverslips containing calculi-containing fecal excreta were analyzed by birefringence microscopy. * denotes p<0.05, two-way ANOVA, Scheffe a correction. FIG. 3g) Incubation of nephrolithiasis patient urine samples (n=3) with 1 mM arbutin at pH7/37° C. for 24 hours showed a significant reduction in birefringence events. In contrast, this difference in birefringence events is not significant in control, healthy urine samples after treatment with arbutin under the same conditions.

FIG. 4 shows arbutin and its interactions with calcium and oxalate according to another embodiment of the present invention. FIG. 4a) Scanning electron microscopy image of arbutin complexed with calcium. EDX spectra reveals 4 calcium ions for every molecule of oxalate. FIG. 4b) Isothermal titration calorimetric analysis of arbutin and calcium chloride, revealing a molar ratio of 4 calcium ions for each arbutin molecule. FIG. 4c) Matrix assisted laser desorption ionization (MALDI) spectrum of calcium and arbutin complexes formed in solution. FIG. 4d) MALDI spectrum of Arbutin and oxalate complexes formed in solution.

FIG. 5 shows calcium oxalate and arbutin crystal structure interaction analysis according to another embodiment of the present invention. FIG. 5a) Confocal birefringence images of pure oxalate crystals prior to Arbutin exposure (left), and following exposure to Arbutin (right). Scale bars are 10 μm. FIG. 5b) Atomic force microscopy (AFM) image pure oxalate crystals. Inset image (middle panels) provides higher magnification of the crystal surface. Scan line analysis of the height channel within the inset box reveals a smooth topography. FIG. 5c) Oxalate crystals exposed to Arbutin reveal a highly active surface topography decorated with arbutin drug molecules (arrows). Inset image (middle panels) reveal a rough surface topography as shown by scan line analysis of the height channel.

FIG. 6 shows cytotoxicity of arbutin and oxalate on human kidney epithelial cells according to another embodiment of the present invention. FIG. 6a) Confocal fluorescence images of HEK293 cells stained with CellTracker Red to label the cell surface and Hoechst to label the nuclei. HEK293 cells were treated with 20 μM sodium oxalate for 30 mins. Birefringence signal was observed within HEK293 cells (white signal). FIG. 6b) Activity of LDH released from HEK293 cells after 30 min incubation with oxalate in the presence or absence of arbutin. FIG. 6c) Cell viability in arbutin treated HEK293 and PC3MLN4 cells over a 3 day period. Green line represents control normalized to 100% at that same timepoint. *** denotes p<0.05, two way ANOVA; Scheffe a correction.

### DETAILED DESCRIPTION

Despite recent advances in surgical care, current understanding of the basic mechanisms that govern nephrolithiasis remains limited. Progress has been limited perhaps due to a lack of suitable pre-clinical models that reliably recapitulate the pathophysiology of this disorder. Several animal models have previously been established for the study of nephrolithiasis including rat, mouse, porcine and canine models. Historically, the most prominent amongst these has been the rat model of nephrolithiasis. The rat model relies on dietary manipulation or intraperitoneal injection of lithogenic agents (ethylene glycol, ammonium chloride or Vitamin D₃) to induce calculus formation. The use of this model has generated variable results with inconsistent stone formation. In addition, the nephrotoxicity of the lithogenic agents has limited overall model utility.

Drosophila melanogaster (D. melanogaster), a species of fly in the family Drosophilidae, has recently emerged as a promising model of human nephrolithiasis. A rapid reproductive rate, a fully mapped and mostly understood genome in tandem with experimental simplicity makes D. melanogaster a particularly powerful model. The renal system of D. melanogaster consists of two discrete components: nephrocytes and Malpighian tubules (MTs). As a unit, these components display a remarkable degree of similarity in form and function to the human nephron. Nephrocytes form a collection of cells around the heart and esophagus that filter waste products from hemolymph in a fashion analogous to the human glomerulus. Said nephrocytes then generate urine by active transport of water, ions and solutes into the Malphigian tubule lumen. Recent work using D. melanogaster has successfully produced calcium oxalate based nephroliths and identified the role of oxalate co-transporters (SLC26A6) and the role of excess zinc in stone formation (Chi T et al. A D. melanogaster Model Identifies a Critical Role for Zinc in Mineralization for Kidney Stone Disease. Singh S R, ed. PLoS One. 2015;10(5):e0124150).

Novel use of imaging techniques is disclosed herein for visualizing and quantifying calcium oxalate stone burden in the D. melanogaster model of calcium oxalate nephrolithiasis. A functional high-throughput screening platform is also disclosed herein. This may allow screening of chemical libraries to identify novel compounds that exhibit anti-lithogenic activity and are ingestible.

Arbutin is currently used as a non-pharmaceutical herbal supplement in the form of a plant-based extract (Bearberry Leaf Dry Extract [BDLE]) for the prevention of urinary tract infections. It is also an active ingredient in skin lightening creams although a risk-benefit approach has been advised with use in this role due a skin sensitization effect. It is also present in significant quantities in wheat based products, pears, coffee and tea. Interestingly, recent studies have associated the regular consumption of coffee and certain teas with a lower risk of stone formation. A higher urinary excretion rate of calcium, decreased calcium oxalate supersaturation and decreased urinary oxalate was seen in caffeinated coffee drinkers.

It is disclosed herein that a specific glycosylated hydroquinone compound of Formula (I), identified using a functional screen of a library of candidate compounds, can bind specifically to oxalate and calcium in both free and bound states. This may lead to disrupting crystal lattice structure, growth and crystallization of nephroliths.

Accordingly, an aspect of the disclosure is a use of a compound of Formula (I) having the structure:

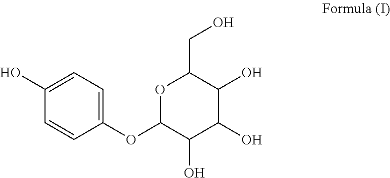 [SMILES: OCC1OC(Oc2ccc(O)cc2)C(O)C(O)C1O]

or a pharmaceutically acceptable salt or solvate thereof, for reducing the size and/or number of calcium oxalate based nephroliths in a subject in need thereof.

In some embodiments, the compound of Formula (I) or a pharmaceutically acceptable salt or solvate thereof is a compound that is used for treating or preventing nephrolithiasis or symptoms associated with nephrolithiasis in the subject.

The term “compound of Formula (I)” as used herein means a glycosylated hydroquinone compound having the structure:

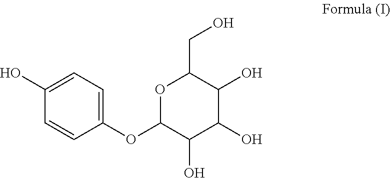 [SMILES: OCC1OC(Oc2ccc(O)cc2)C(O)C(O)C1O]

or pharmaceutically acceptable salts or solvates thereof, as well as mixtures thereof. The compound of Formula (I) is known by the various synonyms including 4-hydroxyphenyl β-D-glucopyranoside, arbutin, hydroquinone β-D-glucopyranoside, hydroquinone-O-beta-D-glucopyranoside, p-hydroxyphenyl β-D-glucopyranoside, p-hydroxyphenyl β-D-glucoside, ursin, uvasol.

As used herein, the term “compound of Formula (I)” is defined to include all forms of the “compound of Formula (I)”, including salts, solvates, hydrates, isomers, stereoisomers, non-crystalline forms, polymorphs, metabolites, as well as any mixtures thereof.

The term “pharmaceutically acceptable salt” means an acid addition salt or basic addition salt that are safe and effective for use in mammals and that possess the desired biological activity. The formation of a desired compound salt is achieved using any standard techniques known in the art. For example, the neutral compound is treated with an acid or base in a suitable solvent and the formed salt is isolated by filtration, extraction or any other suitable method.

Since the “compound of Formula (I)” according to the disclosure herein possesses one or more than one asymmetric centres, they may exist as “stereoisomers”, such as enantiomers and diastereomers. It is to be understood that all such stereoisomers and mixtures thereof in any proportion are encompassed within the scope of the present disclosure. It is to be understood that, while the stereochemistry of the compounds of the disclosure may be as provided for in any given compound shown herein, such compounds may also contain certain amounts (e.g. less than 20%, less than 10%, less than 5%) of compounds having alternate stereochemistry.

The term “subject” as used herein includes all members of the animal kingdom including mammals, and where suitable, refers to humans.

The term “treating” as used herein, unless otherwise indicated, means reversing, alleviating, stabilization (i.e. not worsening) state of disease, delay or inhibiting the progress of, or preventing the disorder or condition to which such term applies, or one more symptoms of such disorder or condition.

The term “treatment”, as used herein, refers to the act of treating, as “treating” is defined immediately above. “Treating” and “treatment” as used herein also include prophylactic treatment. For example, patients at risk for calcium oxalate stone formation could be treated with compound of Formula (I) to promote competitive inhibition, prevent calcium oxalate binding and prevent further stone formation. Moreover, in patients with known calcium oxalate stones that have already formed, destabilization of calcium oxalate crystals could be a mechanism to induce dissolution or enhance the ease of stone fragmentation therapies and prevent progression to chronic kidney stone disease.

The term “nephrolithiasis” as used herein means any condition involving renal calculi, nephroliths or kidney stones that occupy one or more locations in the urinary tract of a subject, e.g. major calices, minor calices, renal pelvis, ureter, etc. Renal calculi or kidney stones can spontaneously develop in the ureter and cause significant health problems, including pain, bleeding, blockage of the ureter, etc.

The term “nephroliths” as used herein refers to stones or calculi that leads to nephrolithiasis.

The term “symptoms associated with nephrolithiasis” as used herein refers to additional conditions experienced by a subject suffering from nephrolithiasis, including but not limited to pain, fever, chills, blood in urine, hypercalcemia, hyperthyroidism, hyperparathyroidism, sarcoidosis, sjogrens syndrome, crohns disease, insulin resistance, acquired renal tubular acidosis, gout, osteoporosis, osteopenia, obesity, overweight, hypertension, anorexia/bulimia, malignancy, urinary dysfunction, abnormal urine odor, urinary leakage; urinary incontinence, urinary leakage, urinary hesitancy, weak urination, urinary blockage, urinary dribbling, nocturnal enuresis, urinary urgency, increased urinary frequency and any combination thereof.

In some embodiments, the use wherein hydroquinone β-D-glucopyranoside or pharmaceutically acceptable salt or solvate thereof is comprised in a pharmaceutical composition that is in a solid, liquid, oral or injectable dosage form.

As used herein, the term “dosage form” refers to the physical form of a compound or composition comprising the compound of Formula (I) or a pharmaceutically acceptable salt or solvate thereof, and includes without limitation liquid and solid dosage forms including, for example tablets, including enteric coated tablets, caplets, gelcaps, capsules, ingestible tablets, buccal tablets, troches, elixirs, suspensions, syrups, wafers, resuspendable powders, functional food composition, food supplement, liquids, solutions as well as injectable dosage forms, including, for example, sterile solutions and sterile powders for reconstitution, and the like, that are suitably Formulated for injection.

Some embodiments disclose a method for evaluating a putative anti-lithogenic agent, the method comprising:
a. inducing a screenably distinct characteristic in wild-type Drosophila by feeding a modified diet;
b. feeding to the Drosophila a compound that putatively modifies the screenably distinct characteristic; and
c. screening and imaging the Drosophila to determine whether the compound modifies the screenably distinct characteristic.

The term “screenably distinct characteristic” as used herein refers to a characteristic of the fly model system for which changes in response to a stimuli can be detected and measured. Example of said characteristic includes growth in size or number of nephroliths in response to the modified diet.

The term “modified diet” as used herein refers to a change in standard diet by addition or substitution or changing amount of additives such as sodium oxalate or calcium chloride.

Another aspect of the present disclosure is a method of identifying a subject with nephrolithiasis likely to benefit from administration of a compound of Formula (I) having the structure

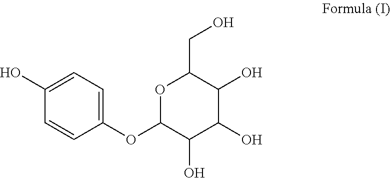 [SMILES: OCC1OC(Oc2ccc(O)cc2)C(O)C(O)C1O]

or a pharmaceutically acceptable salt or solvate thereof, comprising:
a. Obtaining a test sample comprising urine sample from the subject;
b. Determining the calcium oxalate stone burden of the test sample; and
c. Comparing the stone burden of the test sample to urine sample of a control;
   wherein the subject is identified as likely to benefit from the administration of the compound of Formula (I) or pharmaceutically acceptable salt or solvate thereof when the urine sample has an at least 2 fold increased calcium oxalate stone burden compared to the control.

The term “stone burden” as used herein refers to the overall number or size of nephroliths or calculi in the kidney, renal tract or urinary tract of a subject.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the experimental methods and results of the present disclosure:

### EXAMPLES

### Example 1

### Binding Assay of Bisphosphonate Probes to Calcium Oxalate Calculi Formed by D. melanogaster

A. D. Melanogaster Stocks:

All fly stocks were reared on standard medium consisting of agar, yeast, milk powder, corn meal and corn syrup, at 25° C. with a 12-12 hour light-dark cycle at approximately 40% humidity. Wild type Canton S (Bloomington #1) fly line was used for all experiments. For fluorescent tubule imaging, the responder lines UAS-RFP (Bloomington #32218) driven by the GAL4 lines c42 (J. Dow) and URO-GAL4 (Bloomington #44416) were utilised.

B. Lifespan Study:

D. melanogaster eggs were collected on grape juice agar plates in egg collection cages (Diamed Inc.). The eggs were washed multiple times with PBS and 32 mL of the egg solution was pipetted and deposited in vials containing standard food media. After ten days, newly eclosed flies were anesthetized using carbon dioxide and separated by sex. 20 flies of the same gender were added to each of the 10 vials containing varying concentrations of lithogenic agents or standard medium as a control. The flies were transferred to vials containing fresh food on alternate days and the number of deaths recorded. This was done over a period of 60 days. The data was analyzed using the Statistical Package for Social Sciences for Mac (SPSS Version 21 Mac) and graphical data generated using Graph Pad Prism Software (Graphpad Prism 6).

C. Synthesis of Alendronate-FITC and its Negative Isotype Control Notdronate-FITC:

Alendronate-fluorescein isothiocyanate (FITC), a fluorescently labelled bisphosphonate, has been shown to bind to oxalate calculi via petrographic thin sections of calcium oxalate kidney stones and to nanocrystals in patient urine samples. To synthesize alendronate-FITC and notdronate-FITC for the present invention, commercially available bisphosphonate drug alendronate was conjugated to the fluorescent dyes FITC. As a negative control, the bisphosphonate group of alendronate was removed, leaving 4-amino-1-butanol conjugated to the fluorescent dye. The amine (alendronate or 4-amino-1-butanol [Alfa Aesar]) was dissolved in saturated NaHCO₃(aq). The NHS-ester of fluorescein (Thermo Scientific) was dissolved in dimethylformamide (Fisher BioReagents) and added to the amine. The reaction was stirred in the dark overnight. The solution was purified by reverse phase flash column chromatography (Biotage Isolera One, 12 g C18 SNAP, methanol in water 0 to 100%) and the product containing fractions were lyophilized. The lyophilized powder was dissolved in water (Milli-Q,18.2 MΩ·cm), dialyzed with water overnight (Float-A-Lyzer G2, Spectrum Labs), and lyophilized to yield the fluorescent dye conjugates. All reagents were provided in powder form and stored in a −20° C. freezer in an opaque container.

D. Testing Specificity of Fluorescent Probes to Hydroxyapatite and Calcium Based Stones:

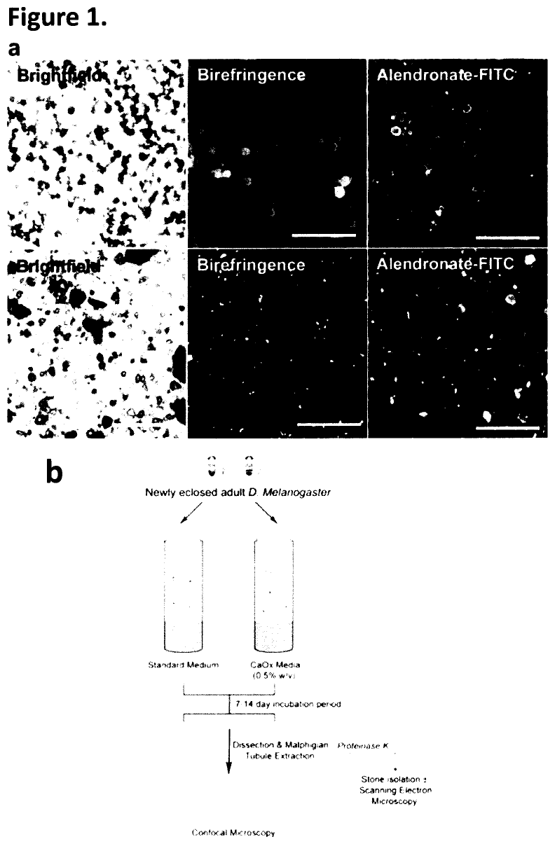

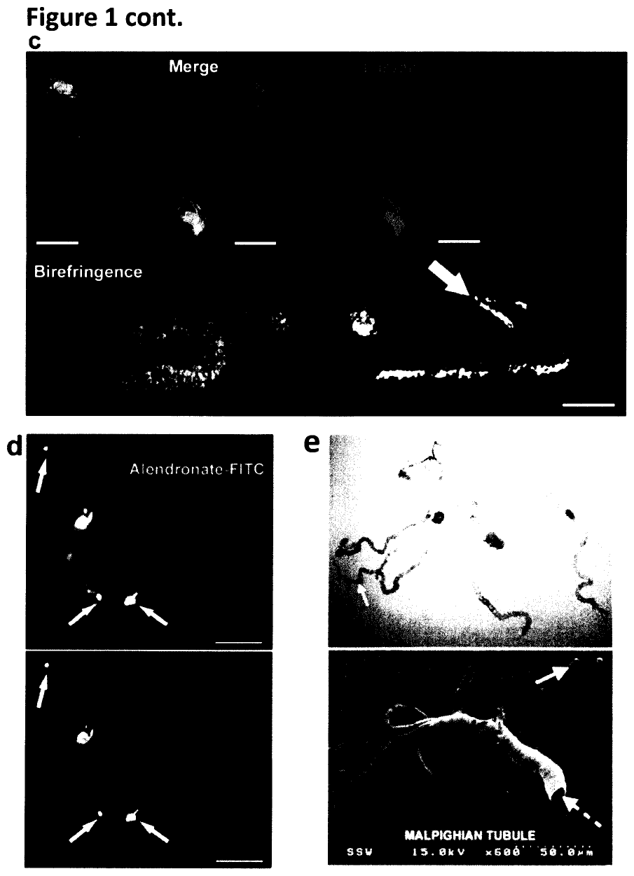

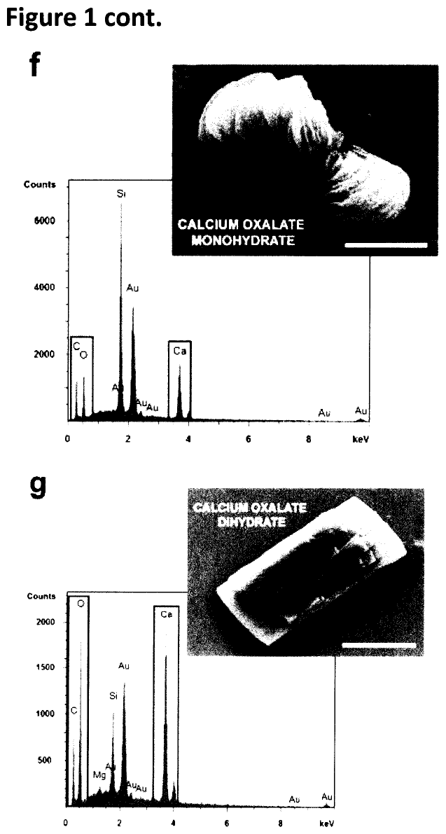

To test the specificity of the fluorescent probes to hydroxyapatite and calcium based stones, pulverized calcium oxalate kidney stones were stained with alendronate-FITC. Hydroxyapatite nanoparticles was used as a positive control to show a high binding affinity of the alendronate-FITC bisphosphonate probe to calcium oxalate calculi. 100 mg of pulverized human calcium oxalate crystals sample was incubated in an Eppendorf tube with 200 mL of 0.1 mM alendronate-FITC, 0.1 mM notdronate-FITC and phosphate-buffered saline (PBS) as a control. Afterwards, the samples were centrifuged (Eppendorf) at 16000 RPM for 5 minutes, the supernatant removed and three subsequent washes performed with distilled water to remove any unbound dye. The samples were then mounted onto glass slides with cover slips for confocal microscopy (Nikon). Referring to FIG. 1a, in a selected embodiment, confocal and birefringence images of pulverized human calcium oxalate crystals (upper row) and synthetic hydroxyapatite particles (lower row) stained with Alendronate-FITC probe is shown.

E. Diet Induced Calcium Oxalate Stone Formation:

Stone forming diets were prepared using the lithogenic agents, sodium oxalate (Sigma Aldrich Inc.) and ethylene glycol (Sigma Aldrich Inc.). These lithogenic agents were added to the water during the preparation of fly food medium. Calcium oxalate calculi were formed in D. melanogaster by supplementing standard fly media with varying amounts of sodium oxalate (0, 0.01, 0.05, 0.1, 0.5, 1.0% w/w) or ethylene glycol (0.1, 1, 2, 5% v/w) and stored in a 4° C. cold room till use. Approximately 20 newly eclosed flies were added to each of 15 vials containing either of the lithogenic diets or standard food medium as a control. Newly eclosed D. melanogaster were added to fly tubes containing supplemented fly media and incubated for 5-14 days. A schematic of D. melanogaster model of calcium oxalate calculi formation within MTs is embodied in FIG. 1b. Following the incubation period, D. melanogaster were removed for staining and imaging.

F. Generation of Fluorescent Malpighian Tubules:

To generate fluorescent MTs in D. melanogaster, the GAL4-UAS system, a potent tool for modifying gene expression, was utilized to develop transgenic fly lines to enhance imaging. Fluorescent MTs were generated by crossing fly lines expressing UAS-GFP and UAS-RFP with the driver lines c42, URO-GAL4 which drive expression in the principal cells of the malpighian tubules and line c724 which drives expression the stellate cells of the malpighian tubules. Age matched newly eclosed virgin females carrying UAS-GFP or UAS-RFP were crossed with newly eclosed males from the driver lines. After 2-3 days of mating and the laying of eggs, the flies were discarded and the transgenic variants were allowed to mature. After maturation, these flies were reared on either the lithogenic diets or the standard control diet for 14 days.

G. Ex Vivo Staining and Imaging of D. Melanogaster Malpighian Tubules:

Flies were euthanized by carbon dioxide narcotization using the Flow Buddy CO2 System (Flystuff Inc.). The malpighian tubules were carefully dissected under a stereomicroscope in a Sylgard (Dow Corning Inc.) lined petri dish using Schneider's media (Sigma Aldrich Inc.). The tubules were mounted on a fresh petri dish and minutien pins (Fine Science Tools) used to anchor the ends. Each tubule was incubated in either 1) 200 mL of 0.1 mM Alendronate-FITC, 2) 0.1 mM Notdronate-FITC or 3) PBS as control for 30 minutes. Consequently, the tubules were washed several times with PBS and then mounted for wide field fluorescence imaging (Nikon Inc.), birefringence microscopy (Nikon Inc.), or resonance confocal microscopy (Nikon Inc.).

For the malpighian tubules expressing either GFP or RFP, they were first stained with 4′,6-diamidino-2-phenylindole (DAPI) dye followed by the fluorescent probes alendronate-FITC and notdronate-FITC of the present invention. The tubules were first imaged with normal light followed by polarized birefringent microscopy and finally imaged with a scanning laser with a confocal microscope. The Nikon TE2000 Inverted Microscope and the Nikon Confocal Microscope were used for imaging.

H. X-Ray Diffraction Spectroscopy:

Approximately 150 flies were dissected. Their malpighian tubules were removed and stored in 1 mL of PBS in an eppendorff tube. The malpighian tubules were then sonicated for 30 minutes in a solution consisting of proteinase K and 0.1% Triton X. Afterwards, they were centrifuged at 16,000 RPM for 10 minutes and the supernatant was discarded. The solid residue was washed three times with distilled water and re-centrifuged. 2 microliters of the sample was mounted on silica wafer for scanning electron microscopy and x-ray diffraction spectroscopy analysis.

Intravital imaging of birefringence signal representing oxalate-based calculi within MTs of D. melanogaster larvae is embodied in FIG. 1c. Larvae with fluorescent MTs, generated using the above methods, were subsequently dissected and imaged for the presence of calculi within their MTs by using D. melanogaster that express the fluorescent protein RFP in the MTs (UAS-RFPxURO-GAL4 cross) (FIG. 1c, upper panels) with birefringence signal representing calculi within the MTs (FIG. 1c, bottom panel, arrow indicates an RFP+ve MT with birefringent signal). MTs positive for birefringence signal were dissected and stained with alendronate-FITC, suggesting formed calculi are calcium oxalate based (FIG. 1d, white arrows). Dissected MTs (FIG. 1e) and their resident calculi were submitted to energy-dispersive X-ray (EDX) spectroscopy, revealing the elemental composition of calculi to be calcium oxalate monohydrate (FIG. 1f) and dihydrate (FIG. 1g). This EDX spectroscopy analysis suggests that D. melanogaster produce calcium oxalate calculi in vivo that are elementally similar to those found in humans.

### Example 2

### D. Melanogaster Birefringence-Positive Fecal Excreta as a High-Throughput Screening Method for Anti-Lithogenic Agents

I. Quantification of Calcium Oxalate Calculi Via D. Melanogaster Excreta Birefringence Assay:

To quantify calcium oxalate calculi via D. melanogaster excreta birefringence assay, approximately 20 newly eclosed flies were added to each of 15 vials containing either of the lithogenic diets or standard food medium as a control. Stone forming diets were prepared according to the method discussed above. A glass coverslip (18×18 mm) was suspended from the cotton tube plug to allow for the collection of fecal matter. Following a 14-day incubation period, the coverslip was extracted from each tube and each coverslip was directly imaged by confocal microscopy under polarized light. Control experiments performed in a similar fashion with standard food media. The percent area of birefringence for each coverslip was individually analyzed and quantified using NIH ImageJ software (Particle Analysis function).

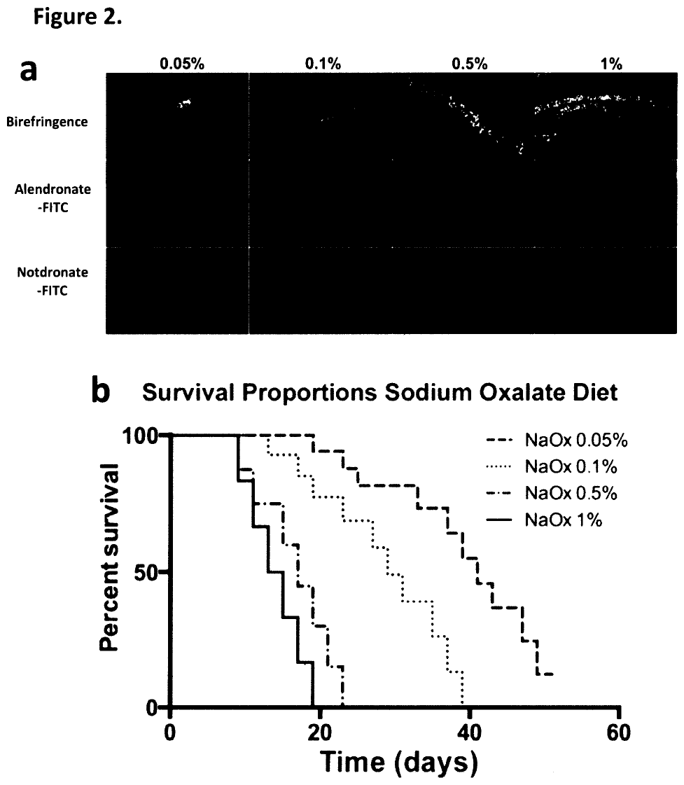

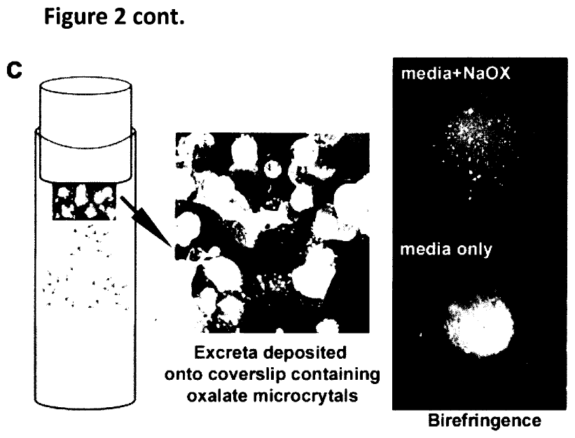

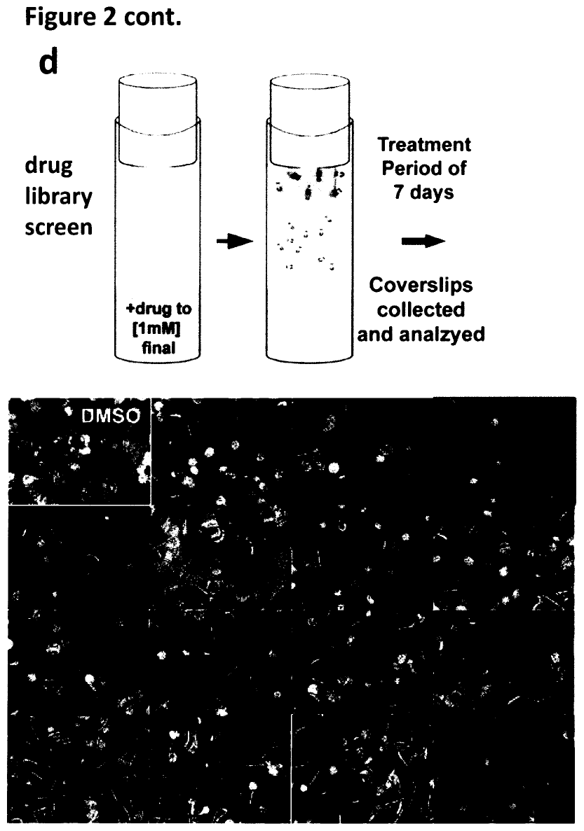

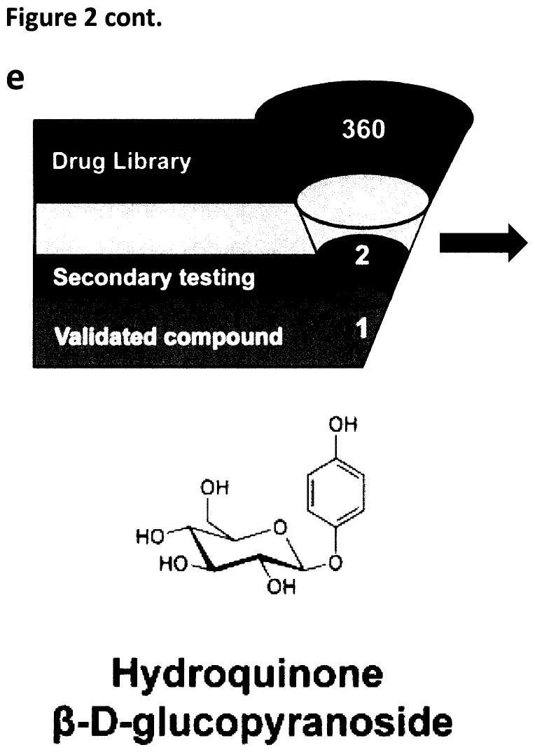

Oxalate based calculi were found throughout the MTs in addition to the fly hindgut. Referring to FIG. 2a, birefringence signals (crystals) and alendronate-FITC staining (calcium oxalate) were utilized to quantitate stone/crystal burden and show that increased concentrations of sodium oxalate added to standard fly media resulted in a proportional increase in stone burden within MTs. However, D. melanogaster survival curve on sodium oxalate treated fly media over a 60-day period show that increasing sodium oxalate concentration impacts fly survival over time (FIG. 2b). Therefore, a 0.5% w/v concentration of sodium oxalate was used in all subsequent experiments. A fecal excreta assay was developed to quantify stone burden by inserting a coverslip in the sponge lid of fly tubes during incubation. D. melanogaster responded by passively depositing calculi-rich fecal excreta onto the coverslip (FIG. 2c, left and middle panels) which was then subjected to polarized light microscopy for birefringence signal representing crystals or calculi. Examination of a single fecal droplet revealed autofluorescence with highly birefringent mineral-like bodies within the fecal deposit. No such birefringent signal was observed in fecal deposits with a standard diet (FIG. 2c, right panels).

J. Drug Library Screening:

A library screen of about 360 candidate compounds was performed in which each candidate compound was mixed into fly media supplemented with 0.5% (w/v) sodium oxalate to a final concentration of 20 μM of candidate compounds. The candidate compounds were screened in duplicates. Five milliliters of the total l0mL media were decanted into two separate 15m1 narrow vials to perform further experiments as duplicates. Approximately 20 newly eclosed flies were added to each vial. Following a 14-day incubation period, each coverslip from the vials was removed and mounted onto glass sides in preparation for imaging. Each slide was directly subjected to confocal microscopy under polarized light. The percentage area of birefringence was quantified and compared birefringence data from control experiments with separately prepared 0.5% sodium oxalate media with no addition of candidate compounds. Hit was defined as coverslips that yielded a <20% reduction in calculi deposition in fecal excreta. Thresholds for inhibition were selected using in vitro analysis with synthetic calcium oxalate crystals.

After 7 days of incubation and ingestion of drug candidates, viability of flies was quantitated and all coverslips containing fecal excreta were analyzed for birefringence signal (FIG. 2d). After analysis of all ˜400 coverslips of which 24 were various negative and positive controls, final analysis yielded a compound “hits” that consistently exhibited anti-lithogenic activity: arbutin (hydroquinone β-D-glucopyranoside) (FIG. 2e).

### Example 3

### Anti-Lithogenic Activity of Arbutin

K. Dose Response Relationship of Arbutin and Calcium Oxalate Stone Formation:

In order to measure dose response relationship of arbutin and calcium oxalate stone formation, thirty milliliters of 0.5% (w/v) sodium oxalate media was prepared with 19,384 μM arbutin. A 10-fold serial dilution was performed to obtain 15 ml of media per dilution. Each dilution was divided into 3 narrow fly vials to yield triplicates. Approximately 20 newly eclosed flies were added to each vial with a glass coverslip (18×18mm) suspended from the cotton via plug to collect fecal matter. After a 14-day incubation period, each coverslip was extracted and stone burden was quantified as described above to observe the dose-response relationship between arbutin concentration and percentage area of birefringence.

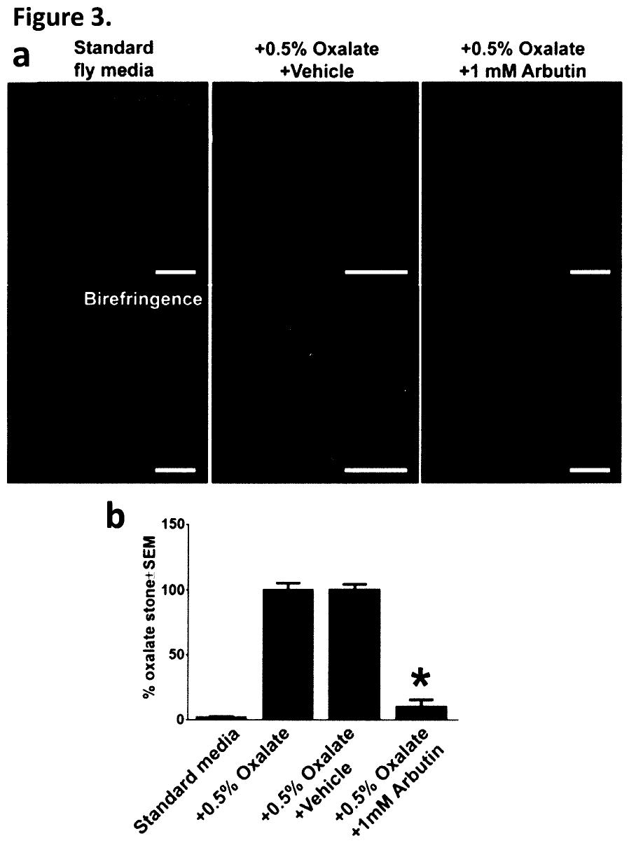

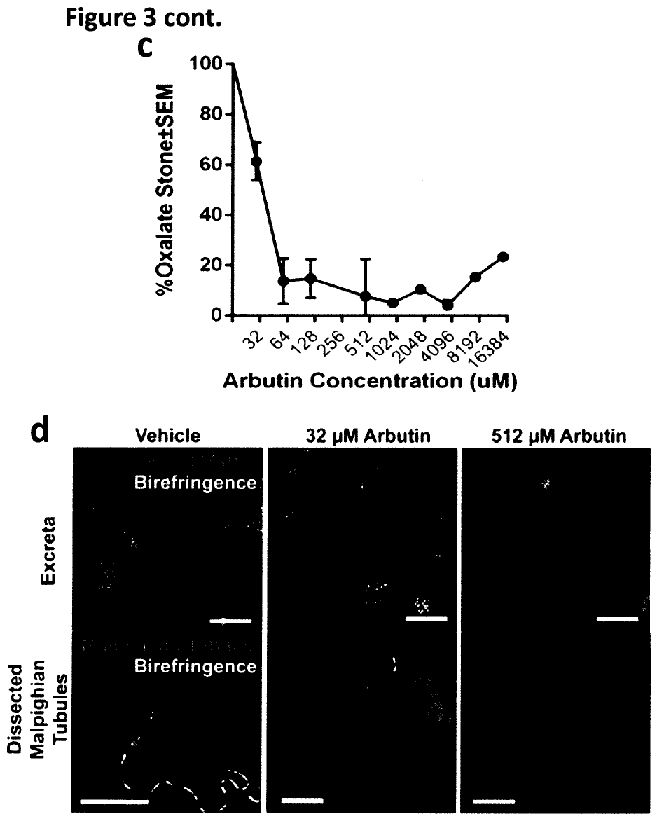

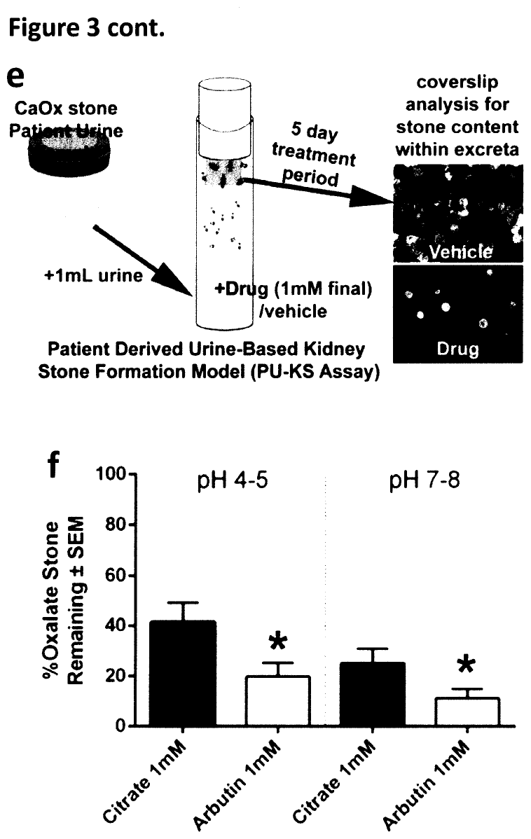

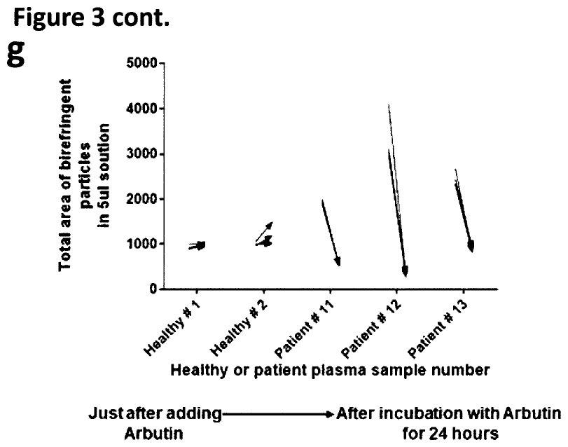

When supplemented to fly media+0.05% sodium oxalate, arbutin at 1 mM resulted in a near abrogation of calculi deposits in dissected MTs when compared to controls (fly media+0.5% oxalate±DMSO) (FIGS. 3a-b). Calculi content deposited in fecal excreta significantly decreased at >64 μM of arbutin (FIG. 3c) with a calculated IC50 of 40 μM. Observed decreases in calculi content in fecal excreta corresponded with decreases in calculi content in MTs with 32 μM and 512 μM arbutin treatment (FIG. 3d).

L. Patient Derived Urine Based Calcium Oxalate Stone Induction in D. Melanogaster:

In addition, effect of arbutin on patient derived urine based calcium oxalate stone induction in D. melanogaster was assessed. Urine samples were collected from recurrent calcium oxalate stone forming patients (n=10 patients) and centrifuged at 2500 rpm. One milliter aliquots of supernatant were prepared and stored at −80° C. for further analysis. Ten milliters of standard medium was prepared and thoroughly mixed with 1mL of patient urine, this was separated into two individual narrow vials (5 mL per vial). Approximately 20 newly eclosed flies were added to each vial. A glass coverslip (18×18 mm) was suspended from the cotton vial plug to collect fecal matter. This experiment was repeated with patient urine containing diet in the presence of 3 mM arbutin. Following a 14-day incubation period, each coverslip was extracted and imaged as described above. Stone burden on each coverslip was quantified via the aforementioned birefringence assay and results were expressed as mean±standard error of mean (SEM). Percentage stone burden were analyzed using one way analysis of variance (ANOVA), (Prism 6, GraphPad Software, Inc., San Diego, Calif., USA) (FIG. 3e).

D. melanogaster incubated in this patient urine based model generated calculi in the same manner as with fly food supplemented with sodium oxalate. This patient urine based model was used to compare the anti-lithogenic effect of arbutin compared to potassium citrate, an established therapy for oxalate-based kidney stone patients. Using 1 mM final concentrations in fly media, arbutin administration lead to a significant decrease in stone burden via fecal excreta assay regardless of pH in comparison with citrate. Various pH's were evaluated in order to recapitulate the pH in human urine at the kidney and bladder (pH 4-6) with a greater reduction in calculi formation observed at pH 7-8 (FIG. 3f, * denotes p<0.05, two-way ANOVA, Scheffe a correction).

M. In Vitro Patient-Urine Based Analysis of Arbutin and Calcium Oxalate Interactions:

50 μl of recurrent calcium oxalate stone forming patient urine and healthy urine samples were combined with 45 μl of 20 mM 4-(2-hydroxyethyl)-1-piperazineethanesulfonic acid (HEPES) buffer (pH 7-8). 5 μl of 20 mM arbutin was added to each solution to form a 1 mM final concentration of the drug. Immediately after addition of the drug, 5 μl of each sample was spotted on a glass slide and birefringence data was imaged on confocal microscope. Each sample was incubated at 37° C. for 24 hours. 5 μl of the sample was identically collected and reimaged. The percent area of birefringence was data analysed as described above. This experiment was repeated in sodium acetate/acetic acid buffer at pH of 4 to observe the efficacy of arbutin in acidic environments. Reduction of percentage stone reduction was analysed using one way ANOVA. Control experiments were conducted using Quick Fix plus synthetic urine from Spectrum labs Cincinnati, Ohio, USA.

Incubation of nephrolithiasis patient urine samples with 1 mM arbutin at pH7/37° C. for 24 hours showed a significant reduction in birefringence events, suggesting that arbutin decreases calcium oxalate stone burden in patient urine samples (FIG. 3g, n=3). In contrast, this difference in birefringence events is not significant in control, healthy urine samples after treatment with arbutin under the same conditions.

### Example 4

### Binding Assay of Arbutin to Both Free Calcium Ions and Oxalate

N. Matrix-assisted Laser Desorption/Ionization (MALDI) of Arbutin-Calcium Chloride and Arbutin-Sodium Oxalate Reaction Mixtures:

To further investigate the interactions of arbutin with calcium and oxalate ions, two separate reaction mixtures containing 1 mM arbutin and 75 mM calcium chloride solution and 1 mM arbutin-75 mM sodium oxalate in 20 mM HEPES buffer (pH 7.4) were prepared. These reaction mixtures were incubated at 37° C. for 24 hours and submitted for mass spectrometry at the Mass Spectrometry Facility, University of Western Ontario, London, Ontario. Samples were analyzed on Bruker Daltonics Reflex IV research grade MALDI time-of-flight (TOF) instrument equipped with a linear/reflectron mass analyzer and post-source decay (PSD).

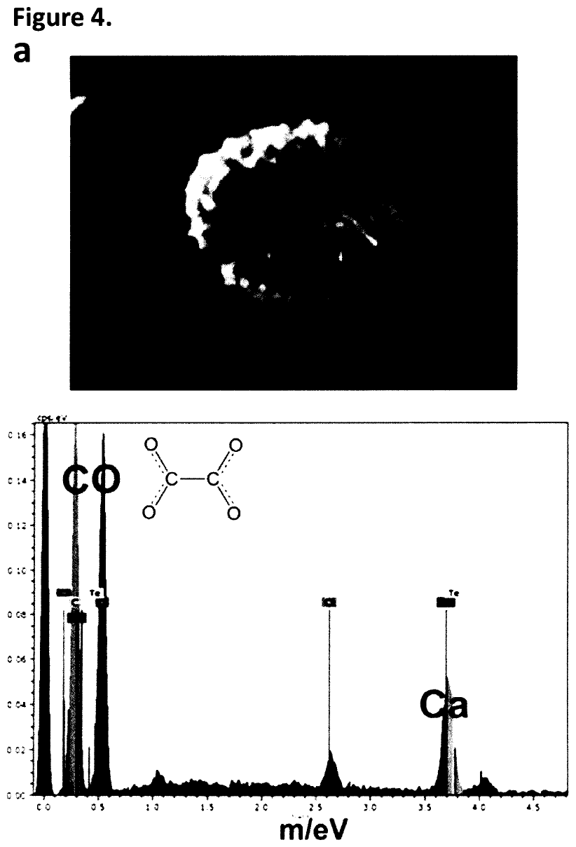

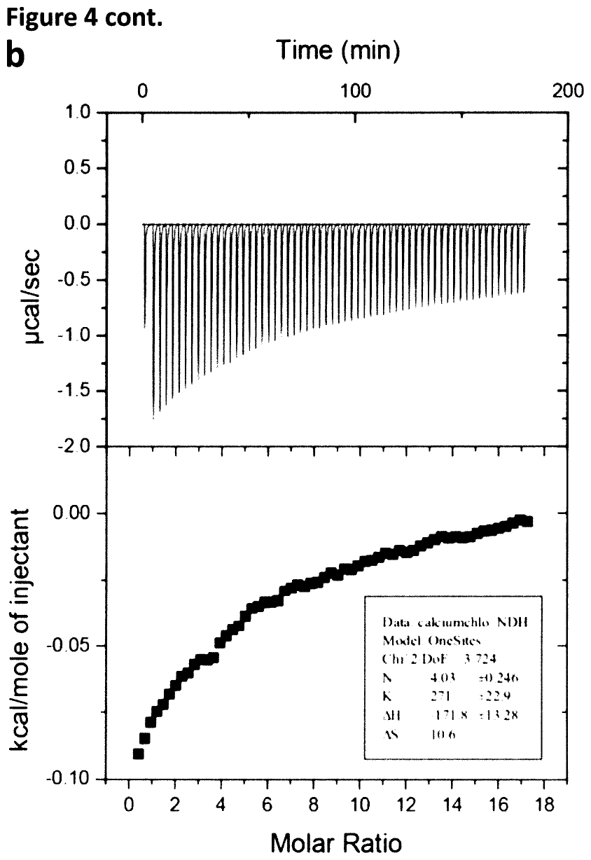

Arbutin, incubated in this manner with calcium chloride and then submitted to scanning electron microscopy, revealed clusters of arbutin and calcium (FIG. 4a, top panel) in a 1:4 stoichiometry as determined by EDX spectroscopy (FIG. 4a, bottom panel).

O. Isothermal Calorimetric Titration of Arbutin and Calcium Chloride:

To investigate the interaction between arbutin and calcium ions, 75 mM calcium chloride and 1 mM arbutin solutions were prepared in 20 mM HEPES buffer at pH 7.4 in preparation for isothermal titration calorimetry (ITC). The ITC binding experiments were performed in the Nano ITC Standard Volume with a fixed gold cell by titrating 250 ul injections of 75 mM calcium chloride solution into 1 mM arbutin solution at 350-second intervals for 1800 seconds (30mins) with stirring speed of 300 revolutions per minute (rpm). The user adjustable auto equilibrate function was used to ensure a stable baseline prior to injection of titrant. ITC data was analyzed with NanoAnalyze Software (TA Instruments).

Isothermal calorimetry of arbutin and calcium chloride revealed an exothermic reaction that plateaued at a molar ratio 4.03 (FIG. 4b). High-performance liquid chromatography (HPLC) and mass spectrometry (MALDI) of arbutin and calcium chloride mixtures revealed peaks representing free arbutin, arbutin complexed with calcium ion and arbutin complexed with calcium ion and water (FIG. 4c).

HPLC and mass spectrometry (MALDI) of arbutin and sodium oxalate also revealed peaks representing arbutin+oxalate+water and arbutin+oxalate formed in solution (FIG. 4d).

### Example 5

### Binding Assay of Arbutin to the Surface of Oxalate-Based Nanocrystals

P. Crystallization of Sodium Oxalate in the Presence and Absence of Arbutin:

To determine the effect of arbutin on the crystallization of calcium oxalate and to determine if arbutin binds directly to the surface of calculi, large sodium oxalate crystals were formed for confocal birefringence microscopy by vapor diffusion. 10 mM sodium oxalate and calcium chloride solutions were prepared in ultrapure water in separate glass vials of different sizes. The smaller glass vial containing the calcium chloride solution was placed inside a larger vial containing sodium oxalate. The system was loosely covered and allowed to rest for a 3 week period with minimal disturbance. This experiment was repeated in the presence of 1 mM arbutin. Crystals formed in the outer oxalate containing vial were collected to for further processing via confocal microscopy and atomic force microscopy.

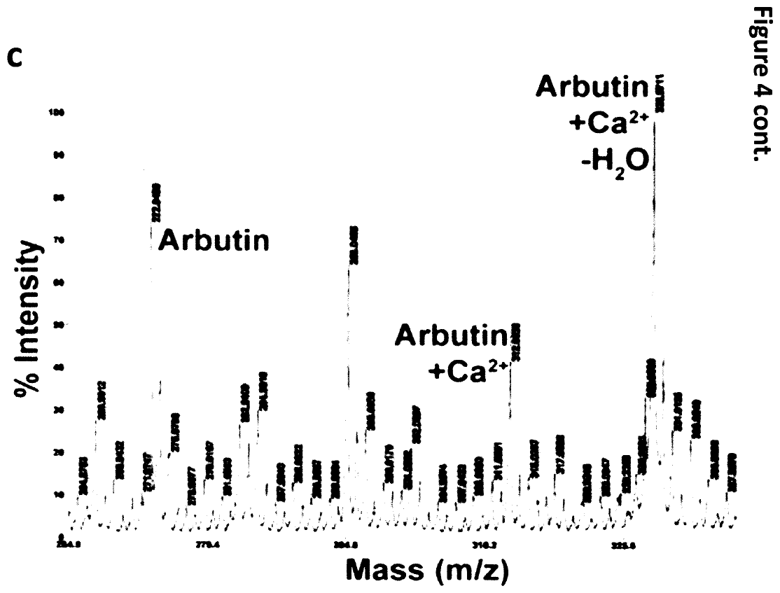

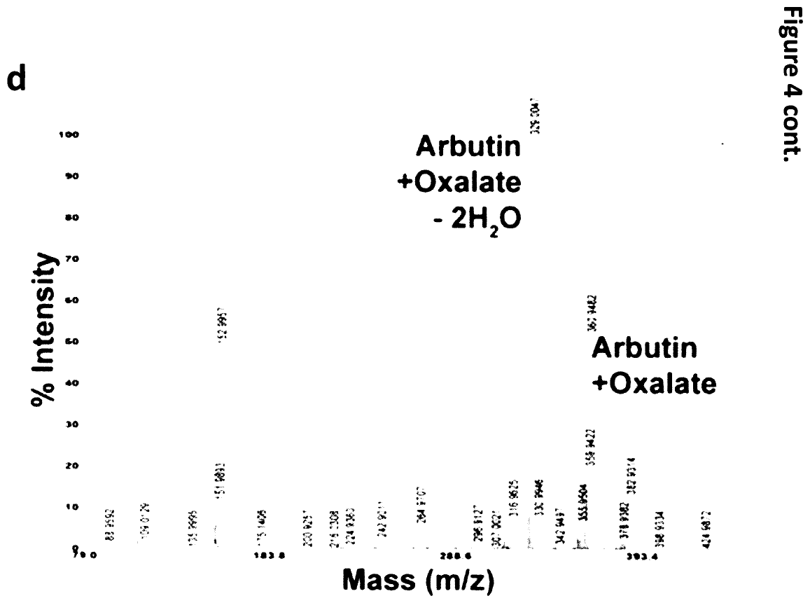

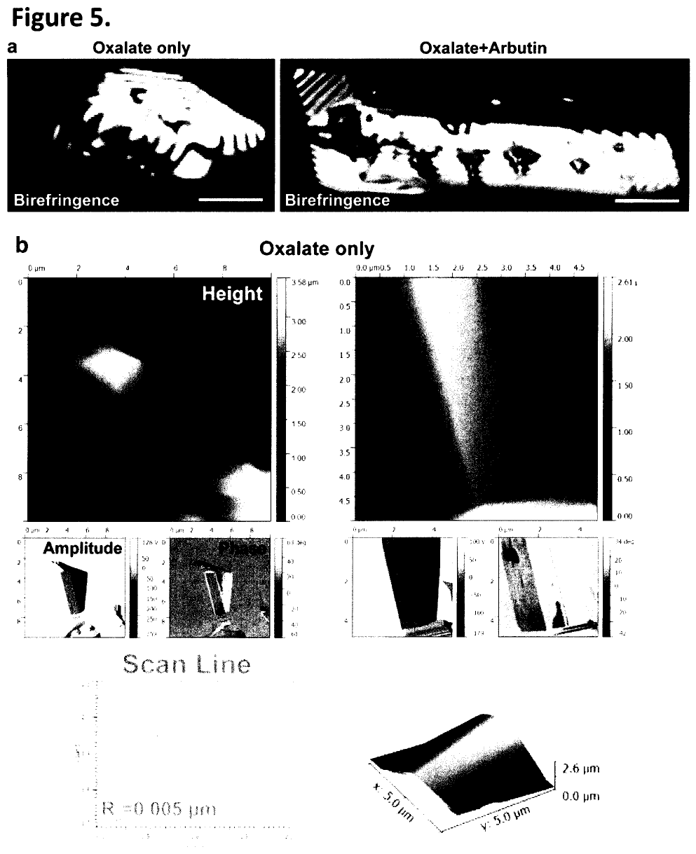

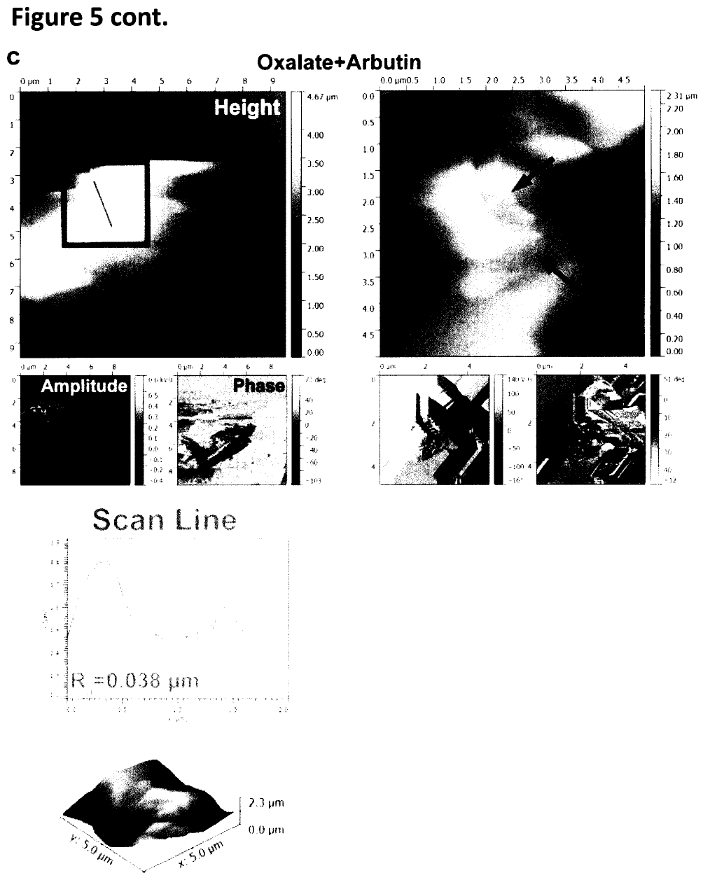

Sodium oxalate calculi imaged in this manner revealed polygonal and smooth surfaces on oxalate-based crystals (FIG. 5a, left panel). Incubation with arbutin resulted in crystal surface aberrations (FIG. 5a, right panel, scale bars are 10 μm). To confirm that arbutin binds to the surface of oxalate-based crystals, atomic force microscopy (AFM) was performed on both crystal types, with oxalate only crystals revealing a smooth and uninterrupted surface according to scan line roughness analysis (FIG. 5b). Oxalate crystals treated with arbutin revealed a highly active crystal topography (FIG. 5c, arrows) with a roughness index higher than in the untreated crystal and a rough surface topography as shown by scan line analysis of the height channel. Oxalate-based crystals were also significantly smaller than non-treated crystals based on AFM volumetric analysis.

### Example 6

### Inhibitory Effect of Arbutin on Oxalate Based Toxicity

Q. Cytotoxicity Assay of Sodium Oxalate and Arbutin on Human Kidney Epithelial Cells:

To assess the cytotoxity of sodium oxalate and arbutin, human embryonic kidney epithelial cells (HEK293; ATCC® CRL-1573™), were cultured in 6-well plate containing 18 mm diameter glass coverslips to 50% confluence. Following aspiration of culture medium, the cells were incubated in a chloride-free solution containing 130 mM Potassium Gluconate, 5 mM Glucose, and 20 mM HEPES (pH 7.4) and 20 μM sodium oxalate for 30 minutes. The treated media was then removed and stored at 4° C. for further analysis. Cells were washed with Cl-free buffer and stained with 20 μM Cell tracker red CMTPX Dye (ThermoFisher Scientific) for 15 mins. Cells were triple washed with Cl-buffer and coverslips were mounted with ProLong® Gold Antifade Mountant with DAPI (ThermoFisher Scientific) without permeabilizing in preparation for confocal microscopy. Samples were imaged using a Nikon A1R+ confocal microscope (1.2 au) with a 20× dry or 60× oil-immersion lens and presented using NIS Elements software (Nikon).

The cytotoxicity of HEK293 cells in sodium oxalate media with or without arbutin was determined by quantifying lactose dehydrogenase (LDH) content liberated by damaged cells using Cytotoxicity Detection KitPLUS (LDH) (Roche) following the manufacturers instructions. Absorbance readings were taken at 492 nm using BioTek PowerWave HT Microplate Spectrophotometer.

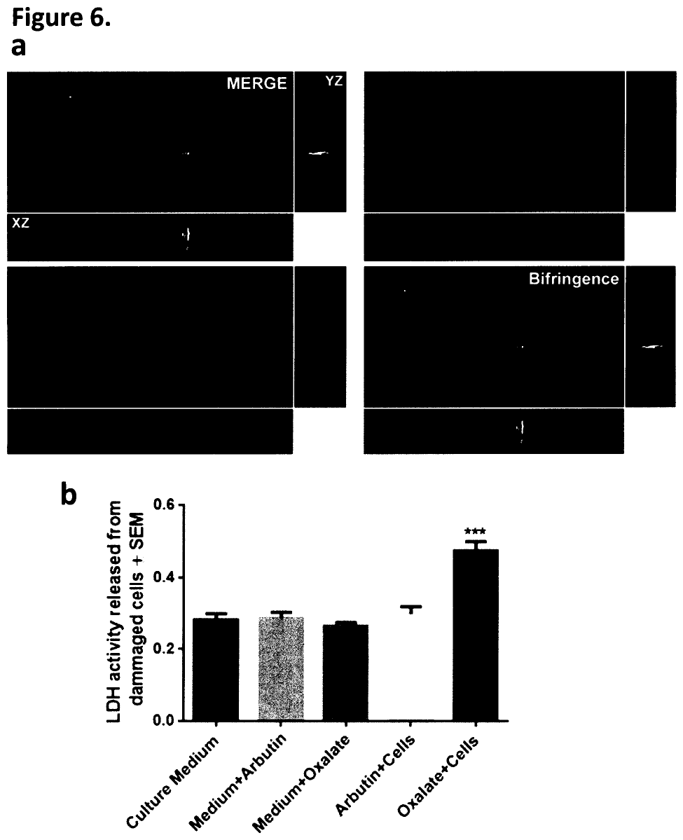

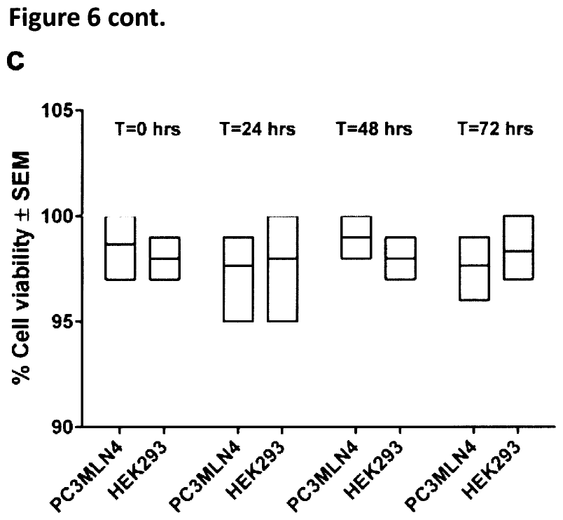

HEK cells grown in vitro under 0.1% calcium oxalate conditions resulted in significantly lower cell viability rates with some nanocrystals observed within cells over the incubation period (FIG. 6a). However, addition of arbutin (1 mM final) into the media ameliorated this effect, with no impact on cell viability or LDH activity compared to arbutin treatment alone (FIGS. 6b-c). Horizontal line in FIG. 6c represents control normalized to 100% at that same timepoint. *** denotes p<0.05, two way ANOVA; Scheffe a correction. No birefringent signal was observed within these cells.

### Discussion:

Aside from the divalent ions, calcium and magnesium, there are currently no known sequestrants for free and bound oxalate, the principal component of calcium oxalate calculi. It is disclosed herein that D. melanogaster as a pre-clinical model of nephrolithiasis may have some value as a high-throughput screening platform for identifying small molecules that inhibited calcium oxalate calculus formation in vivo due to the predominantly acellular metabolic and biochemical nature of nephrolithiasis. This screening platform relies on the dietary administration of candidate drugs to observe changes in calculus composition via the described fecal excreta assay. It can permit an unbiased “drop out” screen to be performed for each candidate drug when ingested. In addition, the use of a natural compound library may be utilized, which focus the screen to compounds that would not elicit a nephrotoxic effect, a current drawback of standard therapies of calculi. As discussed herein, screening a library of 360 different small molecules led to the identification of arbutin as an anti-lithogenic agent.

The pharmacokinetic profile of arbutin is partially established; it is reported to be heavily absorbed from the GI tract and is bioavailable as hydroquinone (HQ) by-products. In previous human studies, 4 hrs following an oral dose of arbutin, approximately 70% of the administered arbutin dose was retrieved in the urine as HQ conjugates, demonstrating renal excretion. The remainder could potentially be accounted for as free arbutin, available to interact with formed calcium oxalate and free subcomponents in the urine. No data on the plasma and urine bioavailability of free arbutin is presently available, possibly due to detection limitations of high performance liquid chromatography (HPLC) based assays utilized in prior studies.

Arbutin has been described to readily degrade into the split components D-glucose and HQ (and conjugates) in acidic environments, an enteric coated tablet vehicle could potentially be utilised to bypass hydrolysis by stomach acids and improve oral bioavailability. However, preliminary nuclear magnetic resonance spectroscopy (NMR) studies of arbutin in acidic conditions disclosed herein suggest that arbutin is stable and able to bind to oxalate whilst in solution despite the low pH. From available toxicology data, arbutin has been shown to have a low risk of acute and chronic toxicity by oral dosing in rat and mouse based experiments with no remarkable effects observed. No genotoxic effects were reported in in vitro or in vivo studies.

The mechanism of arbutin's anti-lithogenic action was evaluated on in vitro calcium oxalate crystals, calculi formed by D. melanogaster, and calculi induced in D. melanogaster by dietary supplementation of patient urine content. Without being limited to any particular theory, the mechanism of action appears to be two fold: arbutin binds to 4 calcium ions and it can bind to oxalate in a 1:1 stoichiometric relationship. The ability of arbutin to interact directly with oxalate is novel since only divalent ions such as calcium have been previously shown to interact with oxalate. Experiments with fly media supplemented with sodium oxalate (0.5%) revealed anti-lithogenic abilities at concentrations as low as 64 μM, suggesting that it directly binds to oxalate, as confirmed by mass spectrometry. arbutin also binds directly to pre-formed oxalate-based calculi as determined by atomic force microscopy and mass spectrometry disrupting crystal lattice structure. Therefore, it appears that arbutin's anti-lithogenic activity is due to its binding to key components of calculi: divalent ions (calcium and magnesium) and oxalate. Translationally, the mode of action of arbutin could be binding to soluble oxalate in the gastrointestinal tract or in the blood.

The observed effects of arbutin in these experiments suggest the potential use of this agent to prevent and or treat pre-existing human calcium oxalate stone disease. In patients at risk for calcium oxalate stone formation, arbutin could through competitive inhibition, prevent calcium oxalate binding. Moreover, in patients with known calcium oxalate stones that have already formed, destabilization of calcium oxalate crystals could be a mechanism to induce dissolution or enhance the ease of stone fragmentation therapies. The latter effect would be a major advancement in the management of calcium oxalate stones as no therapy currently exists to do this.

The fecal excreta calculi assay performed via the D. melanogaster model of human nephrolithiasis may be used as an in vivo screening platform for the identification of anti-lithogenic compounds. It is flexible and scalable to screening of thousands of candidate compounds at minimal cost. The use of birefringent signals to quantitate stone burden via fecal excreta deposited onto coverslips may also be cost effective and may not require any additional processing or staining. It also entails that other birefringent calculi, such as uric acid calculi may also be amenable to this type of chemical library screen. This screening platform provides several avenues of opportunity to narrow down large numbers of potentially overlooked candidate compounds with possible clinical applications for further evaluation.

The above described embodiments of the invention are intended to be illustrative only and in no way limiting. The described embodiments of the invention are susceptible to many modifications of form, arrangement of parts, details and order of operation. The invention, rather, is intended to encompass all such modification within its scope, as defined by the claims.

### REFERENCES:

1. López M, Hoppe B. History, epidemiology and regional diversities of urolithiasis. Pediatr Nephrol. 2010;25(1):49-59. doi:10.1007/s00467-008-0960-5.
2. Romero V, Akpinar H, Assimos D G. Kidney Stones: A Global Picture of Prevalence, Incidence, and Associated Risk Factors. Rev Urol. 2010;12(2-3):e86-e96. http://www.ncbi.nlm.nih.gov/pmc/articles/PMC2931286/.
3. Scales C D, Smith A C, Hanley J M, Saigal C S, Urologic Diseases in America Project. Prevalence of kidney stones in the United States. Eur Urol. 2012;62(1):160-165. doi:10.1016/j.eururo.2012.03.052.
4. El-Zoghby Z M, Lieske J C, Foley R N, et al. Urolithiasis and the Risk of ESRD. Clin J Am Soc Nephrol. 2012;7(9):1409-1415. doi:10.2215/CJN.03210312.
5. Kovshilovskaya B, Chi T, Miller J, Stoller M L. Systemic implications of urinary stone disease. Transl Androl Urol. 2012;1(2):89-96. doi:10.3978/j.issn.2223-4683.2012.06.05.
6. Lotan Y, Cadeddu J, Roerhborn C, Pak C, Pearle M. Cost-effectiveness of medical management of strategies for nephrolithiasis. J Urol. 2004;172(6):2275-2281. doi:10.1097/01.ju.0000141498.11720.20.
7. Katsuma S, Shiojima S, Hirasawa A, et al. Global analysis of differentially expressed genes during progression of calcium oxalate nephrolithiasis. Biochem Biophys Res Commun. 2002;296(3):544-552. http://www.ncbi.nlm.nih.gov/pubmed/12176015.
8. Finkielstein VA. Strategies for preventing calcium oxalate stones. Can Med Assoc J. 2006;174(10):1407-1409. doi:10.1503/cmaj.051517.
9. Pak CY. Kidney stones. Lancet. 1998;351(9118):1797-1801. doi:10.1016/S0140-6736(98)01295-1.
10. Reilly R F, Peixoto A J, Desir G V. The Evidence-Based Use of Thiazide Diuretics in Hypertension and Nephrolithiasis. Clin J Am Soc Nephrol. 2010;5(10):1893-1903. doi:10.2215/CJN.04670510.
11. Qaseem A, Dallas P, Forciea M A, Starkey M, Denberg T D. Dietary and Pharmacologic Management to Prevent Recurrent Nephrolithiasis in Adults: A Clinical Practice Guideline From the American College of Physicians. Ann Intern Med. 2014;161(9):659. doi:10.7326/M13-2908.
12. Rattan V, Sidhu H, Vaidyanathan S, Thind S K, Nath R. Effect of combined supplementation of magnesium oxide and pyridoxine in calcium-oxalate stone formers. Urol Res. 1994;22(3): 161-165. http://www.ncbi.nlm.nih.gov/pubmed/7992461.
13. Balcke P, Schmidt P, Zazgornik J, Kopsa H, Minar E. Pyridoxine therapy in patients with renal calcium oxalate calculi. Proc Eur Dial Transplant Assoc. 1983;20:417-421. http://www.ncbi.nlm.nih.gov/pubmed/6657665.
14. Mitwalli A, Ayiomamitis A, Grass L, Oreopoulos D G. Control of hyperoxaluria with large doses of pyridoxine in patients with kidney stones. Int Urol Nephrol. 1988;20(4):353-359. http://www.ncbi.nlm.nih.gov/pubmed/3170105.
15. Moe O W, Pearle M S, Sakhaee K. Pharmacotherapy of urolithiasis: evidence from clinical trials. Kidney Int. 2011;79(4):385-392. doi:10.1038/ki.2010.389.
16. Xu H, Zisman A L, Coe F L, Worcester E M. Kidney stones: an update on current pharmacological management and future directions. Expert Opin Pharmacother. 2013;14(4):435-447. doi:10.1517/14656566.2013.775250.
17. Khan S R. Animal models of kidney stone formation: an analysis. World J Urol. 1997;15(4):236-243. doi:10.1007/BF01367661.
18. Liu J, Cao Z, Zhang Z, Zhou S, Ye Z. A comparative study on several models of experimental renal calcium oxalate stones formation in rats. J Huazhong Univ Sci Technolog Med Sci. 2007;27(1):83-87. doi:10.1007/s11596-007-0124-z.
19. Khan S R, Glenton P A, Byer K J. Modeling of hyperoxaluric calcium oxalate nephrolithiasis: experimental induction of hyperoxaluria by hydroxy-L-proline. Kidney Int. 2006;70(5):914-923. doi:10.1038/sj.ki.5001699.
20. Khan S R, Glenton P A. Experimental Induction of Calcium Oxalate Nephrolithiasis in Mice. J Urol. 2010;184(3):1189-1196. doi:10.1016/j.juro.2010.04.065.
21. Miller J, Chi T, Kapahi P, et al. D. melanogaster melanogaster as an Emerging Translational Model of Human Nephrolithiasis. J Urol. 2013;190(5):1648-1656. doi:10.1016/j.juro.2013.03.010.
22. Weavers H, Prieto-Sanchez S, Grawe F, et al. The insect nephrocyte is a podocyte-like cell with a filtration slit diaphragm. Nature. 2009;457(7227):322-326. doi:10.1038/nature07526.
23. Hirata T, Cabrero P, Berkholz D S, et al. In vivo Drosophilia genetic model for calcium oxalate nephrolithiasis. AJP Ren Physiol. 2012;303(11):F1555-F1562. doi:10.1152/ajprenal.00074.2012.
24. Chi T, Kim MS, Lang S, et al. A D. melanogaster Model Identifies a Critical Role for Zinc in Mineralization for Kidney Stone Disease. Singh S R, ed. PLoS One. 2015;10(5):e0124150. doi:10.1371/journal.pone.0124150.
25. Carmen POP, Laurian VLASE M T. Natural Resources Containing arbutin. Determination of arbutin in the Leaves of Bergenia crassifolia (L.) Fritsch. acclimated in Romania. Not Bot Horti Agrobot Cluj-Napoca. 2009;(37.1):129-132.
26. Gavin C T, Ali S N, Tailly T, et al. Novel Methods of Determining Urinary Calculi Composition: Petrographic Thin Sectioning of Calculi and Nanoscale Flow Cytometry Urinalysis. Sci Rep. 2016;6:19328. doi:10.1038/srep19328.
27. Duffy JB. GAL4 system inD. melanogaster: A fly geneticist's swiss army knife. genesis. 2002;34(1-2):1-15. doi:10.1002/gene.10150.
28. Schindler G, Patzak U, Brinkhaus B, et al. Urinary excretion and metabolism of arbutin after oral administration of Arctostaphylos uvae ursi extract as film-coated tablets and aqueous solution in healthy humans. J Clin Pharmacol. 2002;42(8):920-927. http://www.ncbi.nlm.nih.gov/pubmed/12162475.
29. O'Donoghue JL. Hydroquinone and its analogues in dermatology—a risk-benefit viewpoint. J Cosmet Dermatol. 2006;5(3):196-203. doi:10.1111/j.1473-2165.2006.00253.x.
30. Ferraro P M, Taylor E N, Gambaro G, Curhan G C. Soda and other beverages and the risk of kidney stones. Clin J Am Soc Nephrol. 2013;8(8):1389-1395. doi:10.2215/CJN.11661112.
31. Ferraro P M, Taylor E N, Gambaro G, Curhan G C. Caffeine intake and the risk of kidney stones. Am J Clin Nutr. 2014;100(6):1596-1603. doi:10.3945/ajcn.114.089987.
32. Chemical Information Review Document for arbutin [CAS No. 497-76-7] and Extracts from Arctostaphylos uva-ursi. Support Nomin Toxicol Eval by Natl Toxicol Progr. 2006.
33. Glockl I, Blaschke G, Vei M. Validated methods for direct determination of hydroquinone glucuronide and sulfate in human urine after oral intake of bearberry leaf extract by capillary zone electrophoresis. J Chromatogr B Biomed Sci Appl. 2001;761(2):261-266. http://www.ncbi.nlm.nih.gov/pubmed/11587357.
34. Deisinger P J, Hill T S, English J C. Human exposure to naturally occurring hydroquinone. J Toxicol Environ Health. 1996;47(1):31-46. doi:10.1080/009841096161915.
35. Products) S (Scientific C on C. Opinion on β-arbutin. Dir 76/768/ECC, CAS 497-76-7, EINECS 207-850-3.

1. Use of a compound having the structure of Formula (I): 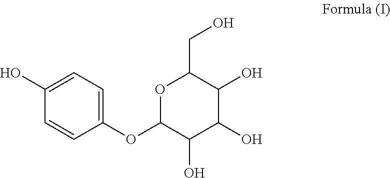 [SMILES: OCC1OC(Oc2ccc(O)cc2)C(O)C(O)C1O]
or a pharmaceutically acceptable salt or solvate thereof, for reducing the size and/or number of calcium oxalate based nephroliths in a subject in need thereof.

2. The use of claim 1 for treating or preventing nephrolithiasis in the subject.

3. The use of claim 1 for treating or preventing symptoms associated with nephrolithiasis in the subject.

4. The use of any of claims 1 to 3, wherein hydroquinone β-D-glucopyranoside or pharmaceutically acceptable salt or solvate thereof is comprised in a pharmaceutical composition.

5. The use of claim 4, wherein the pharmaceutical composition comprises one or more suitable excipients, diluents, buffers, carriers or vehicles.

6. The use of claim 4 or 5, wherein the pharmaceutical composition is in a solid, liquid, oral or injectable dosage form.

7. The use of claims 4 to 6, wherein the pharmaceutical composition is administered by parenteral, subcutaneous, intravenous, intraperitoneal, transdermal, oral, buccal, intravaginal, intravesicular, depot injection or implants.

8. The use of any one of claims 4 to 7, wherein the hydroquinone β-D-glucopyranoside is administered with an additional agent selected from the group consisting of ketorolac, acetaminophen, ibuprofen, aspirin, xanthine oxidase inhibitor, potassium citrate, potassium magnesium, magnesium citrate, neutral (nonacidic) sodium, potassium phosphate, cellulose phosphate, cholestyramine, tamsulosin, tiopronin, diuretics, hydrochlorothiazide, chlorothiazide, trichlormethiazide, chlorthalidone, amiloride, citrate salts, phosphates, cholestyramine, sodium bicarbonate, aluminum hydroxide anti-acid gel, acetohydroxamic acid, allopurinol, penicillamine, captopril, nonsteroidal anti-inflammatory drugs (NSAIDs) and any combination thereof.

9. The use of claim 3, wherein the symptom is selected from pain, fever, chills, blood in urine, hypercalcemia, hyperthyroidism, hyperparathyroidism, sarcoidosis, sjogrens syndrome, crohns disease, insulin resistance, acquired renal tubular acidosis, gout, osteoporosis, osteopenia, obesity, overweight, hypertension, anorexia/bulimia, malignancy, urinary dysfunction, abnormal urine odor, urinary leakage; urinary incontinence, urinary leakage, urinary hesitancy, weak urination, urinary blockage, urinary dribbling, nocturnal enuresis, urinary urgency, increased urinary frequency and any combination thereof.

10. The use of any one of claims 1 to 9, wherein the amount of hydroquinone β-D-glucopyranoside ranges from about 50 mg to 850 mg/day.

11. A method for evaluating a putative anti-lithogenic agent, the method comprising:
a. inducing a screenably distinct characteristic in wild-type Drosophila by feeding a modified diet;
b. feeding to the Drosophila a compound that putatively modifies the screenably distinct characteristic; and
c. screening and imaging the Drosophila to determine whether the compound modifies the screenably distinct characteristic.

12. A method according to claim 14 wherein the screenably distinct characteristic comprises formation of calcium oxalate based nephroliths.

13. A method according to claim 14 wherein the modified diet comprises stone forming media with sodium oxalate.

14. A method according to claim 14 further comprising screening the Drosophila to determine whether the compound has a toxic effect on the Drosophila.

15. A method of identifying a subject with nephrolithiasis likely to benefit from administration of a compound of Formula (I) having the structure 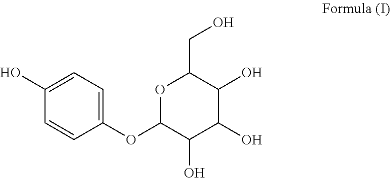 [SMILES: OCC1OC(Oc2ccc(O)cc2)C(O)C(O)C1O]
or a pharmaceutically acceptable salt or solvate thereof, comprising:
a. Obtaining a test sample comprising urine sample from the subject;
b. Determining the calcium oxalate stone burden of the test sample; and
c. Comparing the stone burden of the test sample to urine sample of a control;
wherein the subject is identified as likely to benefit from the administration of the compound of Formula (I) or pharmaceutically acceptable salt or solvate thereof when the urine sample has an at least 2 fold increased calcium oxalate stone burden compared to the control.

16. A kit comprising a compound of Formula (I) having the structure 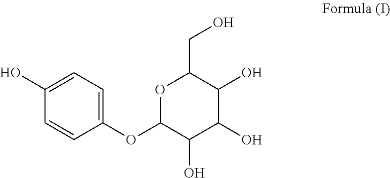 [SMILES: OCC1OC(Oc2ccc(O)cc2)C(O)C(O)C1O]
or a pharmaceutically acceptable salt or solvate thereof, optionally another agent, and/or packaging instructions for use thereof.
